(19) United States
(12) Patent Application Publication
(10) Pub. No.: US 2017/0020565 A1
(43) Pub. Date: Jan. 26, 2017

(54) AUTOMATIC HAIR TRANSPLANTER FOR TRANSPLANTING FOLLICLES

(71) Applicants: Electronics and Telecommunications Research Institute, Daejeon (KR); Kyungpook National University Industry-Academic Corporation Foundation, Daegu (KR)

(72) Inventors: Tae Wuk BAE, Daegu (KR); Kyu Hyung KIM, Daegu (KR); Moon Kyu KIM, Daegu (KR); Jung Chul KIM, Daegu (KR); Jung Wook SUH, Daegu (KR); Soo In LEE, Daejeon (KR); Hyung Soo LEE, Daegu (KR); Yong Chul JUNG, Daegu (KR); Eun Chang CHOI, Daegu (KR); Dae Sik KIM, Daejeon (KR); Chang Hyuk HONG, Daegu (KR)

(73) Assignees: Electronics and Telecommunications Research Institute, Daejeon (KR); Kyungpook National University Industry-Academic Corporation Foundation, Daejeon (KR)

(21) Appl. No.: 15/138,433

(22) Filed: Apr. 26, 2016

(30) Foreign Application Priority Data
Jul. 22, 2015 (KR) 10-2015-0103799

(51) Int. Cl. A61B 17/34; A61B 17/3205

(52) U.S. Cl. CPC A61B 17/3468; A61B 17/32053; A61B 2017/00752

(57) ABSTRACT

Provided is an automatic hair transplanter for transplanting follicles including a housing having a space therein and having a nozzle in which an opening is formed on a front surface, a needle supply magazine installed on the housing and sequentially supplying a plurality of needles containing follicles to the back of the nozzle, a needle aligner transferring and aligning a needle supplied from the needle supply magazine with the center of the nozzle, a needle actuator causing the needle aligned by the needle aligner to protrude from the nozzle, a follicle pressurizer pressurizing a follicle in the needle protruding by the needle actuator from the back to the front, a needle retrieval magazine containing the needle in which pressurization of the follicle has been finished by the follicle pressurizer and nothing remains, and a manipulator having a switch for controlling operation of the needle actuator and the follicle pressurizer.

### CROSS-REFERENCE TO RELATED APPLICATION

This application claims priority to and the benefit of Korean Patent Application No. 10-2015-0103799, filed on Jul. 22, 2015, the disclosure of which is incorporated herein by reference in its entirety.

### BACKGROUND

1. Field of the Invention

The present invention relates to a hair transplanter for transplanting follicles, and more particularly, to an automatic hair transplanter for transplanting follicles which enables a reduction in the time taken for follicle transplantation and also the minimization of transplantation operator fatigue in a follicle transplantation process.

2. Discussion of Related Art

A large number of people suffer from alopecia, that is, excessive loss of hair, such as the hair on one's head. Here, a region of hair loss is remarkably shown compared to a region of no hair loss. Therefore, some alopecia patients wear wigs for beauty, thereby preventing regions of hair loss from being remarkably shown.

However, it is inconvenient to wear and manage a wig, and a wig does not harmonize with existing hair and thus is followed by the sense of difference. Also, a region on which a wig can be put is limited.

For these reasons, alopecia patients who have hair transplantation in regions of hair loss are increasing lately.

Since hair transplantation results in long-term maintenance of the effect of covering a region of hair loss and no sense of difference from existing hair, the number of alopecia patients who consider hair transplantation is constantly increasing.

Meanwhile, a manual hair transplanter for transplanting follicles is generally used in a hair transplantation procedure. An existing manual hair transplanter for transplanting follicles includes a needle containing a follicle and a rod for pressurizing the follicle contained in the needle. In the process of inserting the needle containing the follicle into skin and then withdrawing the needle, the follicle is transplanted into the skin by pressurizing the follicle inserted into the skin with the rod.

Using such an existing manual hair transplanter for transplanting follicles, it is possible to transplant a follicle in a region of hair loss. However, only one follicle is transplanted through one needle, and thus it takes a considerable time to transplant hundreds to thousands of follicles in a region of hair loss.

Also, in the case of the existing manual hair transplanter for transplanting follicles, a body containing the needle is lifted by a transplantation operator's hand work, that is, with the transplantation operator's thumb, index finger, etc., to withdraw the needle. Therefore, when transplanting a large number of follicles, the transplantation operator feels considerable fatigue due to repeated hand works, and transplantation operators who work for a long period may suffer from a musculoskeletal disease.

For the aforementioned reasons, development of an automatic hair transplanter for transplanting follicles which enables a reduction in the time taken for follicle transplantation and also the minimization of transplantation operator fatigue in a follicle transplantation process is under way in the corresponding field, but satisfactory results have not been obtained so far.

### SUMMARY OF THE INVENTION

The present invention is directed to providing an automatic hair transplanter for transplanting follicles which enables a reduction in the time taken for follicle transplantation and also the minimization of transplantation operator fatigue in a follicle transplantation process.

According to an aspect of the present invention, there is provided an automatic hair transplanter for transplanting follicles, the automatic hair transplanter including: a housing having a space therein and having a nozzle in which an opening is formed on a front surface; a needle supply magazine installed on the housing and configured to sequentially supply a plurality of needles containing follicles to a back of the nozzle; a needle aligner configured to transfer and align a needle supplied from the needle supply magazine with a center of the nozzle; a needle actuator configured to cause the needle aligned by the needle aligner to protrude from the nozzle; a follicle pressurizer configured to pressurize a follicle in the needle protruding by the needle actuator from a back to a front; a needle retrieval magazine configured to contain the needle in which pressurization of the follicle has been finished by the follicle pressurizer and nothing remains; and a manipulator having a switch for controlling operation of the needle actuator and the follicle pressurizer.

The housing may be formed to be divisible into an upper part and a lower part.

The needle supply magazine may have an elastic member supporting the plurality of stacked needles downward in an upper end portion thereof.

The needle aligner may include a pair of support parts disposed to face each other, and a pair of guides guiding each of the support parts.

The guides may have guide grooves including inclined portions and parallel portions on upper surfaces.

The needle actuator may include a motor configured to rotate upon application of power, a screw configured to rotate by rotation of the motor, a moving block threadedly engaged with the screw and configured to move forward or backward according to the rotation of the screw, a connection shaft coupled to the moving block and configured to move forward or backward together with the moving block, a gripper coupled to a front end of the connection shaft and configured to grip and fix the needle, and a coupling plate coupled to the connection shaft and connected to the guides of the needle aligner.

A center of the connection shaft may correspond to the center of the nozzle.

The coupling plate may have bumps inserted into the guide grooves provided in the guides of the needle aligner.

The follicle pressurizer may include a motor configured to rotate upon application of power, a screw configured to rotate by rotation of the motor, a moving block threadedly engaged with the screw and configured to move forward or backward according to the rotation of the screw, and a rod coupled to the moving block and configured to move forward or backward together with the moving block.

A front end of the rod may penetrate a center of the connection shaft of the needle actuator.

The needle retrieval magazine may be disposed under the needle aligner.

The manipulator may be disposed on an outer surface of the housing.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a perspective view of an automatic hair transplanter for transplanting follicles according to an exemplary embodiment of the present invention;

FIG. 2 is an exploded perspective view illustrating a structure of an automatic hair transplanter for transplanting follicles according to an exemplary embodiment of the present invention;

FIG. 3 is an example diagram illustrating supply of a needle through a needle supply magazine according to an exemplary embodiment of the present invention;

FIG. 4 is an example diagram illustrating alignment of a needle through a needle aligner according to an exemplary embodiment of the present invention;

FIG. 5 is an example diagram illustrating protrusion of a needle through a needle actuator according to an exemplary embodiment of the present invention;

FIG. 6 is an example diagram illustrating pressurization of a follicle through a follicle pressurizer according to an exemplary embodiment of the present invention; and

FIG. 7 is an example diagram illustrating retrieval of a needle through a needle retrieval magazine according to an exemplary embodiment of the present invention.

### DETAILED DESCRIPTION OF EXEMPLARY EMBODIMENTS

Advantages and features of the present invention and a method of achieving the same will be more clearly understood from embodiments described below in detail with reference to the accompanying drawings. However, the present invention is not limited to the following embodiments and may be implemented in various different forms. The embodiments are provided merely for complete disclosure of the present invention and to fully convey the scope of the invention to those of ordinary skill in the art to which the present invention pertains. The present invention is defined only by the scope of the claims.

Meanwhile, the terminology used herein is for the purpose of describing the embodiments and is not intended to be limiting of the invention. As used in this specification, the singular form of a word includes the plural unless the context clearly indicates otherwise. The term “comprise” and/or “comprising,” when used herein, does not preclude the presence or addition of one or more components, steps, operations, and/or elements other than stated components, steps, operations, and/or elements.

Hereinafter, an automatic hair transplanter for transplanting follicles according to an exemplary embodiment of the present invention will be described in detail with reference to the accompanying drawings.

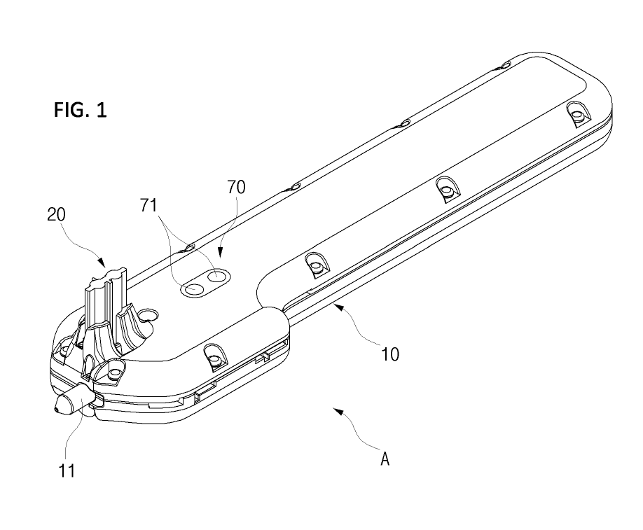

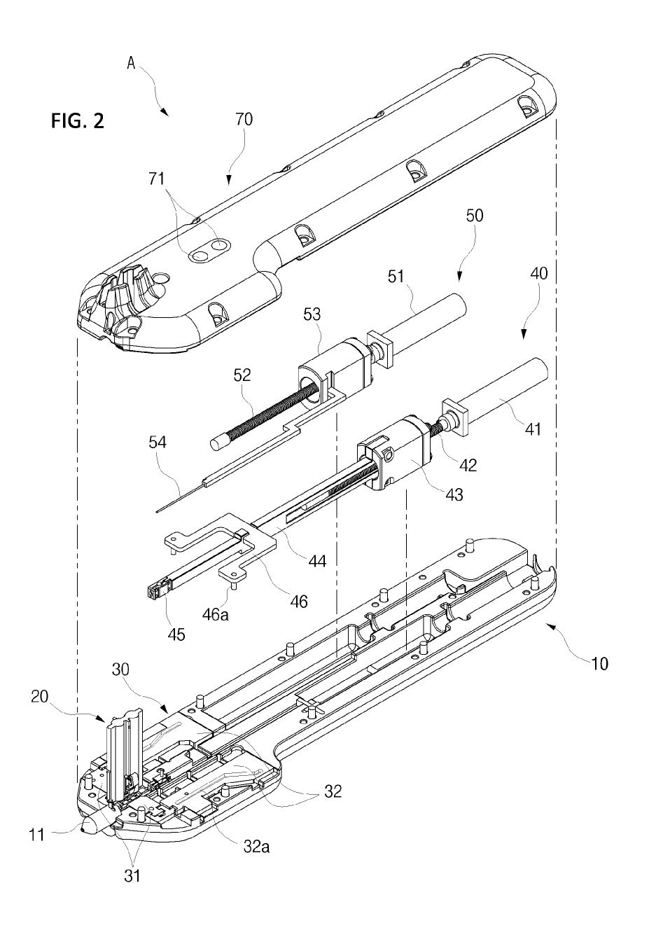

As shown in FIGS. 1 and 2, an automatic hair transplanter A for transplanting follicles according to an exemplary embodiment of the present invention includes a housing 10, a needle supply magazine 20, a needle aligner 30, a needle actuator 40, a follicle pressurizer 50, an needle retrieval magazine 60, and a manipulator 70.

The housing 10 has a space therein, and has a nozzle 11 having an opening (not shown) formed on a front surface.

The housing 10 is formed to be divisible into an upper part and a lower part.

Since the housing 10 is formed to be divisible into the upper part and the lower part, when the housing 10 is divided into the upper part and the lower part, the needle aligner 30, the needle actuator 40, and the follicle pressurizer 50 may be disposed in the housing 10.

The needle supply magazine 20 is installed on the housing 10 and sequentially supplies a plurality of needles 100 containing follicles 200 to a back of the nozzle 11.

This needle supply magazine 20 has an elastic member 21 supporting the plurality of stacked needles 100 downward in an upper end portion thereof.

Since the needle supply magazine 20 has the elastic member 21 supporting the plurality of stacked needles 100 downward in the upper end portion thereof, a needle 100 is released from the needle supply magazine 20 by pressurization of the elastic member 21 and supplied to the back of the nozzle 11.

Meanwhile, the needle supply magazine 20 has a blocking part 22 which prevents a lowermost needle 100 among the plurality of needles 100 from being released to the outside thereof.

Since the needle supply magazine 20 has the blocking part 22 which prevents the lowermost needle 100 among the plurality of needles 100 from being released to the outside, the needle 100 is supplied from the inside of the needle supply magazine 20 to the back of the nozzle 11 only when the blocking part 22 is open. Here, the blocking part 22 is open only at the moment of coming in contact with one of support parts 31 which will be described below when the support parts 31 are moved close to each other, so that the needles 100 are not released from the needle supply magazine 20 at a time but are released one after another.

The needle aligner 30 transfers the needle 100 supplied from the needle supply magazine 20 and aligns the needle 100 with the center of the nozzle 11.

This needle aligner 30 includes one pair of the support parts 31 disposed to face each other and one pair of guides 32 which guide each of the support parts 31.

Since the needle aligner 30 includes the pair of support parts 31 disposed to face each other and the pair of guides 32 which guide each of the support parts 31, when the pair of support parts 31 are moved to close to each other with movement of the guides 32, the needle 100 between the pair of guides 32 is fixed. Also, when the needle 100 fixed by the support parts 31 is moved together with the support parts 31 and a front end of the needle 100 is disposed right behind the nozzle 11, the needle 100 is aligned with the center of the nozzle 11.

The guides 32 have guide grooves 32a which include inclined portions (not shown) and parallel portions (not shown) on the upper surfaces thereof.

Since the guides 32 have the guide grooves 32a which include the inclined portions and the parallel portions on the upper surfaces, a coupling plate 46 which will be described below moves forward or backward with bumps 46a of the coupling plate 46 inserted into the guide grooves 32a. Since the bumps 46a are in contact with the guide grooves 32a, the guides 32 do not only move lengthwise but also move apart from or close to each other. Accordingly, the support parts 31 may move lengthwise and apart from or close to each other.

The needle actuator 40 causes the needle 100 aligned by the needle aligner 30 to protrude from the nozzle 11.

This needle actuator 40 includes a motor 41 which rotates upon application of power, a screw 42 which rotates by rotation of the motor 41, a moving block 43 which is threadedly engaged with the screw 42 and moves forward or backward according to rotation of the screw 42, a connection shaft 44 which is coupled to the moving block 43 and moves forward or backward together with the moving block 43, a gripper 45 which is coupled to a front end of the connection shaft 44 and grips and fixes the needle 100, and a coupling plate 46 which is coupled to the connection shaft 44 and connected to the guides 32 of the needle aligner 30.

Since the needle actuator 40 includes the motor 41 rotating upon application of the power, the screw 42 rotating by the rotation of the motor 41, the moving block 43 threadedly engaged with the screw 42 and moving forward or backward according to the rotation of the screw 42, the connection shaft 44 coupled to the moving block 43 and moving forward or backward together with the moving block 43, the gripper 45 coupled to the front end of the connection shaft 44 and gripping and fixing the needle 100, and the coupling plate 46 coupled to the connection shaft 44 and connected to the guides 32 of the needle aligner 30, when the connection shaft 44 moves forward, the needle 100 passes through the nozzle 11 and protrudes from the housing 10, and also the support parts 31 and the guides 32 of the needle aligner 30 may operate. Here, the center of the connection shaft 44 corresponds to the center of the nozzle 11.

Since the center of the connection shaft 44 corresponds to the center of the nozzle 11, when the connection shaft 44 moves forward, a back end of the needle 100 aligned with the center of the nozzle 11 is accurately gripped and fixed by the gripper 45 coupled to the front end of the connection shaft 44, and also the needle 100 gripped and fixed by the gripper 45 passes through the nozzle 11 and protrudes from the housing 10.

The coupling plate 46 has the bumps 46a inserted into the guide grooves 32a.

Since the coupling plate 46 has the bumps 46a inserted into the guide grooves 32a, when the bumps 46a are inserted into the guide grooves 32a, the coupling plate 46 and the guides 32 are connected, and the guides 32 are operated by forward or backward movement of the coupling plate 46.

After the needle 100 is withdrawn, the gripper 45 releases the fixed needle 100.

Since the gripper 45 releases the fixed needle 100 after the needle 100 is withdrawn, the released needle 100 in which nothing remains after pressurization of a follicle 200 may fall to the needle retrieval magazine 60 thereafter.

The follicle pressurizer 50 pressurizes the follicle 200 in the needle 100 protruding by the needle actuator 40 from the back to the front.

This follicle pressurizer 50 includes a motor 51 which rotates upon application of power, a screw 52 which rotates by rotation of the motor 51, a moving block 53 which is threadedly engaged with the screw 52 and moves forward or backward according to rotation of the screw 52, and a rod 54 which is coupled to the moving block 53 and moves forward or backward together with the moving block 53.

Since the follicle pressurizer 50 includes the motor 51 rotating upon application of the power, the screw 52 rotating by the rotation of the motor 51, the moving block 53 threadedly engaged with the screw 52 and moving forward or backward according to the rotation of the screw 52, and the rod 54 coupled to the moving block 53 and moving forward or backward together with the moving block 53, when the rod 54 moves forward, the follicle 200 in the needle 100 is pressurized from the back to the front. Here, a front end of the rod 54 penetrates the center of the connection shaft 44.

Since the front end of the rod 54 penetrates the center of the connection shaft 44, the front end of the rod 54 moving forward together with the moving block 53 is directed to the center of the needle 100 fixed by the gripper 45 coupled to the front end of the connection shaft 44, and the follicle 200 contained in the needle 100 is pressurized by contact with the front end of the rod 54 from the back to the front.

The needle retrieval magazine 60 contains the needle 100 in which pressurization of the follicle 200 has been finished by the follicle pressurizer 50 and nothing remains.

This needle retrieval magazine 60 is disposed under the needle aligner 30.

Since the needle retrieval magazine 60 is disposed under the needle aligner 30, when the support parts 31 of the needle aligner 30 are moved apart from each other, it is possible to contain the empty needle 100 falling down.

The needle retrieval magazine 60 may employ any structure and any method for containing a plurality of empty needles 100 falling down from the needle aligner 30.

The manipulator 70 has switches 71 for controlling operation of the needle actuator 40 and the follicle pressurizer 50.

This manipulator 70 is disposed on an outer surface of the housing 10.

Since the manipulator 70 is disposed on the outer surface of the housing 10, it is easy to manipulate the manipulator 70 while grabbing the housing 10.

Transplantation of a follicle 200 through the automatic hair transplanter A for transplanting follicles according to an exemplary embodiment of the present invention will be described in detail below.

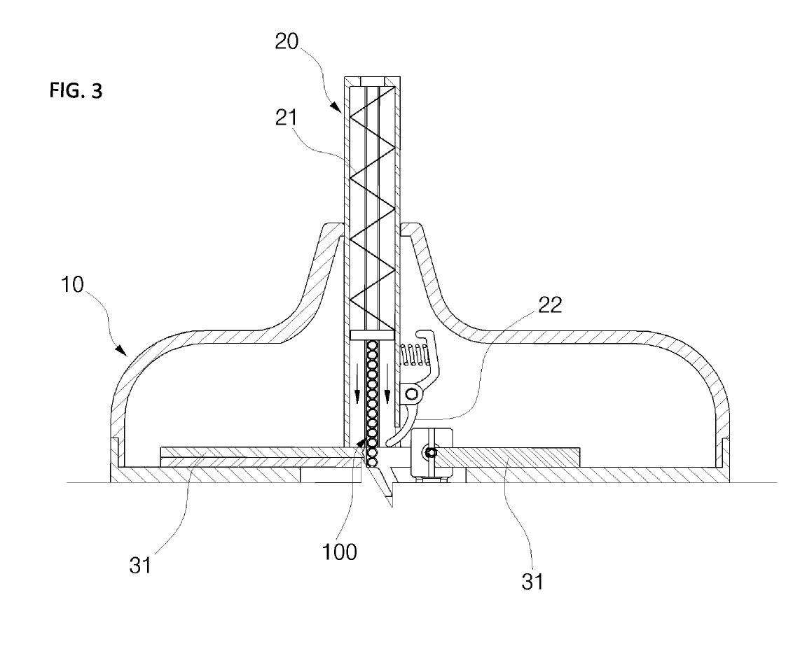

In an exemplary embodiment of the present invention, the needle supply magazine 20 has the elastic member 21 in the upper end portion thereof, and thus the plurality of needles 100 stacked in the needle supply magazine 20 are released downward due to the downward support of the elastic member 21 as shown in FIG. 3.

In an exemplary embodiment of the present invention, since the support parts 31 of the needle aligner 30 are disposed on both sides of a needle 100 supplied from the needle supply magazine 20, the needle 100 supplied from the needle supply magazine 20 is fixed by moving the support parts 31 close to each other.

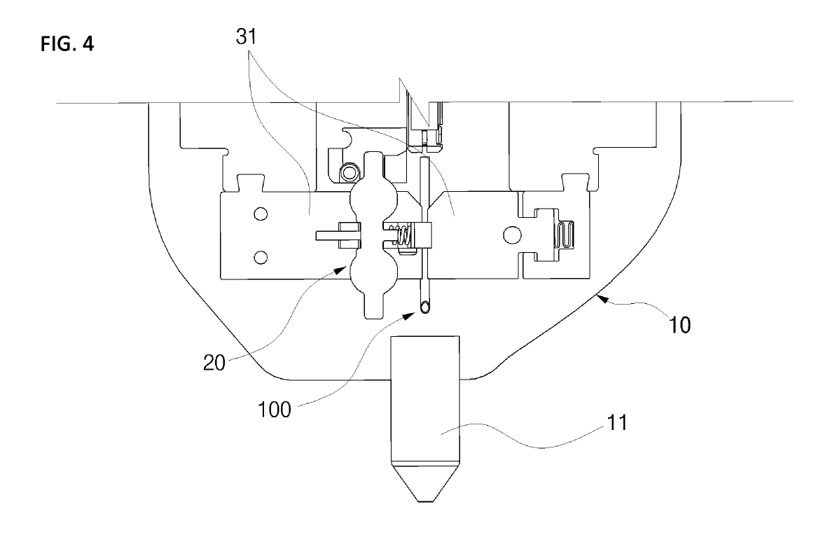

Since the support parts 31 move by operation of the guides 32, the needle 100 fixed by the support parts 31 is moved right behind the nozzle 11 by operating the guides 32, so that the needle 100 is aligned with the center of the nozzle 11 as shown in FIG. 4.

In an exemplary embodiment of the present invention, since the connection shaft 44 of the needle actuator 40 is threadedly engaged with the screw 42 which rotates by the rotation of the motor 41 and moves forward or backward by the rotation of the screw 42, when the connection shaft 44 is moved forward by rotating the motor 41 of the needle actuator 40, the gripper 45 at the front end of the connection shaft 44 grips and fixes the back end of the aligned needle 100.

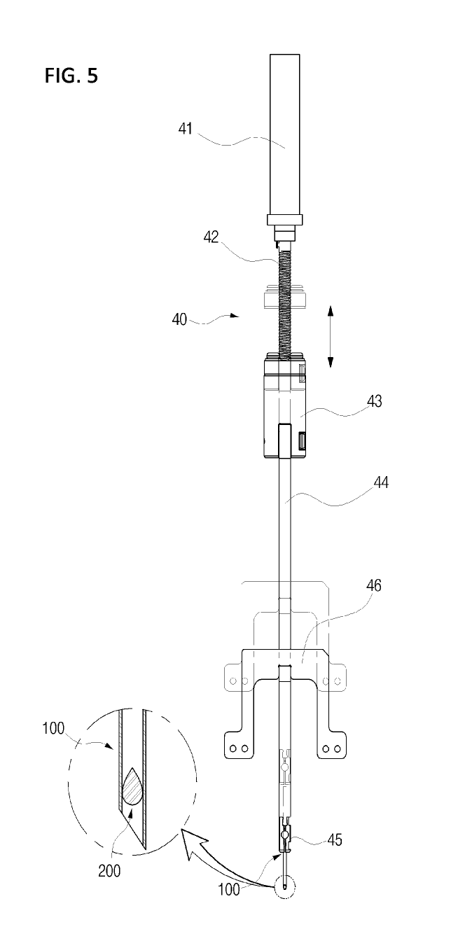

Therefore, when the connection shaft 44 further moves forward while the needle 100 is fixed by the gripper 45, the needle 100 fixed by the gripper 45 passes through the nozzle 11 and protrudes from the housing 10 as shown in FIG. 5, and thus it is possible to insert the needle 100 into the skin of a region of hair loss.

When the connection shaft 44 further moves forward after the gripper 45 grips and fixes the back end of the needle 100, the support parts 31 coming in contact with the side of the needle 100 are moved apart from each other in the process of the bumps 46a of the coupling plate 46 coming in contact with the inclined portions of the guide grooves 32a. Therefore, the needle 100 may protrude from the housing 10 by forward movement of the connection shaft 44.

Meanwhile, while the needle 100 is inserted into the skin, the follicle 200 is still contained in the needle 100. Therefore, when the needle 100 is withdrawn as it is, the follicle 200 is withdrawn together and is not stably embedded in the skin.

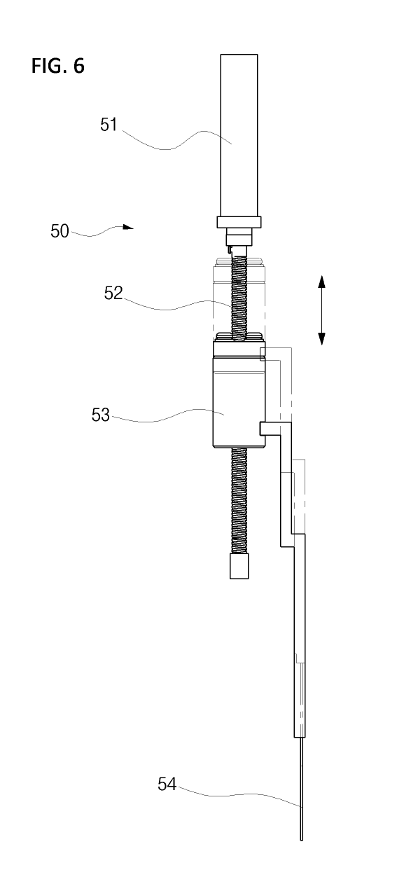

However, according to an exemplary embodiment of the present invention, the follicle pressurizer 50 is provided in the housing 10. When the screw 52 is rotated by operating the motor 51, the moving block 53 threadedly engaged with the screw 52 moves forward, and the rod 54 coupled to the moving block 53 moves forward. Therefore, as shown in FIG. 6, the follicle 200 in the needle 100 is pressurized by contact with the front end of the rod 54 from the back to the front to get out of the front end of the needle 100, and stably embedded in the skin. After that, even when the needle 100 and the rod 54 are withdrawn, the follicle 200 is kept stably embedded.

Therefore, continuous transplantation of follicles 200 is possible by repeating the process of aligning the needles 100 sequentially supplied from the needle supply magazine 20 through the needle aligner 30, inserting an aligned needle 100 into skin through the needle actuator 40, and pressurizing a follicle 200 in the needle 100 inserted into the skin through the follicle pressurizer 50, and it is possible to reduce the time taken to transplant the follicles 200.

In an exemplary embodiment of the present invention, the needle retrieval magazine 60 is disposed under the needle aligner 30.

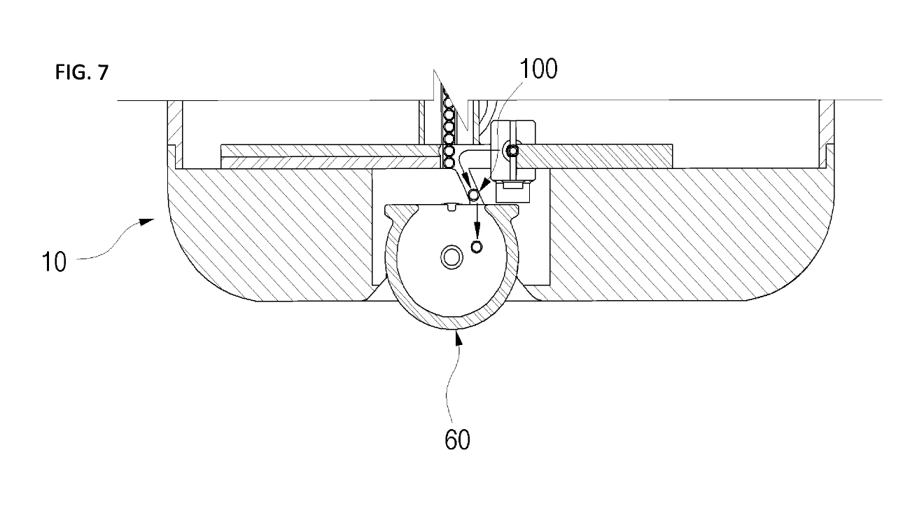

Therefore, as shown in FIG. 7, when the support parts 31 of the needle aligner 30 are moved apart from each other, the needle 100 in which nothing remains after pressurization of the follicle 200 falls down and is contained in the needle retrieval magazine 60, so that the empty needle 100 may be retrieved.

Meanwhile, in an exemplary embodiment of the present invention, the needle actuator 40 and the follicle pressurizer 50 are operated by manipulation of the switches 71 of the manipulator 70. Therefore, in the process of transplanting the follicles 200, a transplantation operator is not required to do a hand work except to manipulate the switches 71 of the manipulator 70. In other words, in the process of transplanting the follicles 200, hand works of the transplantation operator may be minimized, and transplantation operator fatigue is minimized.

According to the inventive automatic hair transplanter A for transplanting follicles, the plurality of needles 100 sequentially supplied from the needle supply magazine 20 are aligned by the needle aligner 30, and then caused by the needle actuator 40 to protrude from the housing 10 and be inserted into skin, and a follicle 200 in a needle 100 inserted into the skin is pressurized by the follicle pressurizer 50 and stably embedded in the skin. Therefore, it is possible to continuously transplant follicles 200 contained in each of the plurality of needles 100. Consequently, it is possible to reduce the time taken to transplant the follicles 200 and also minimize transplantation operator fatigue in the process of transplanting the follicles 200.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

What is claimed is:

1. An automatic hair transplanter for transplanting follicles, the automatic hair transplanter comprising:
a housing having a space therein and having a nozzle in which an opening is formed on a front surface;
a needle supply magazine installed on the housing and configured to sequentially supply a plurality of needles containing follicles to a back of the nozzle;
a needle aligner configured to transfer and align a needle supplied from the needle supply magazine with a center of the nozzle;
a needle actuator configured to cause the needle aligned by the needle aligner to protrude from the nozzle;
a follicle pressurizer configured to pressurize a follicle in the needle protruding by the needle actuator from a back to a front;
a needle retrieval magazine configured to contain the needle in which pressurization of the follicle has been finished by the follicle pressurizer and nothing remains; and
a manipulator having a switch for controlling operation of the needle actuator and the follicle pressurizer.

2. The automatic hair transplanter of claim 1, wherein the housing is formed to be divisible into an upper part and a lower part.

3. The automatic hair transplanter of claim 1, wherein the needle supply magazine has an elastic member supporting the plurality of stacked needles downward in an upper end portion thereof.

4. The automatic hair transplanter of claim 1, wherein the needle aligner includes:
a pair of support parts disposed to face each other; and
a pair of guides configured to guide each of the support parts.

5. The automatic hair transplanter of claim 4, wherein the guides have guide grooves including inclined portions and parallel portions on upper surfaces.

6. The automatic hair transplanter of claim 5, wherein the needle actuator includes:
a motor configured to rotate upon application of power;
a screw configured to rotate by rotation of the motor;
a moving block threadedly engaged with the screw and configured to move forward or backward according to the rotation of the screw;
a connection shaft coupled to the moving block and configured to move forward or backward together with the moving block;
a gripper coupled to a front end of the connection shaft and configured to grip and fix the needle; and
a coupling plate coupled to the connection shaft and connected to the guides of the needle aligner.

7. The automatic hair transplanter of claim 6, wherein a center of the connection shaft corresponds to the center of the nozzle.

8. The automatic hair transplanter of claim 6, wherein the coupling plate has bumps inserted into the guide grooves provided in the guides of the needle aligner.

9. The automatic hair transplanter of claim 6, wherein the follicle pressurizer includes:
a motor configured to rotate upon application of power;
a screw configured to rotate by rotation of the motor;
a moving block threadedly engaged with the screw and configured to move forward or backward according to the rotation of the screw; and
a rod coupled to the moving block and configured to move forward or backward together with the moving block.

10. The automatic hair transplanter of claim 9, wherein a front end of the rod penetrates a center of the connection shaft of the needle actuator.

11. The automatic hair transplanter of claim 1, wherein the needle retrieval magazine is disposed under the needle aligner.

12. The automatic hair transplanter of claim 1, wherein the manipulator is disposed on an outer surface of the housing.
